(19) United States
(12) Patent Application Publication
(10) Pub. No.: US 2020/0069029 A1
(43) Pub. Date: Mar. 5, 2020

(54) Liquid Applicator and Device

(71) Applicant: Porex Corporation, Fairburn, GA (US)

(72) Inventors: Thomas Oliver Knight, III, Newnan, GA (US); Saman Mahdavi Shahidani, Decatur, GA (US); Garland Timberlake Meredith, Peachtree City, GA (US); Xingguo Li, Peachtree City, GA (US); Timothy Martin, Newnan, GA (US); Avi Melech Robbins, Atlanta, GA (US); Guoqiang Mao, Peachtree City, GA (US)

(21) Appl. No.: 16/485,101

(22) PCT Filed: Feb. 19, 2018
(86) PCT No.: PCT/US2018/018637

§ 371 Date: Aug. 9, 2019

Related U.S. Application Data
(60) Provisional application No. 62/460,279, filed on Feb. 17, 2017.

(51) Int. Cl. A45D 34/04; B65D 47/42; A61M 35/00; C08J 9/00; C08J 9/24

(52) U.S. Cl. CPC A45D 34/042; B65D 47/42; A61M 35/003; B29C 67/205; C08J 9/24; A45D 2200/1009; C08J 9/0061

(57) ABSTRACT

The present application provides sintered porous elastomeric liquid applicators with or without flocking fibers that provide improved liquid and gel delivery properties and a comfortable experience for the user of the applicators when applying liquid to a surface, such as skin.

### FIELD OF THE INVENTION

The present invention provides a sintered porous elastomeric liquid applicator that provides improved liquid and gel delivery properties and a comfortable experience for the user of the applicator when applying liquid to a surface.

### BACKGROUND

U.S. Pat. No. 5,899,622 discloses a liquid and semi-liquid applicator with a porous core and flocking on the one end of the applicator. It discloses that the porous core could be sintered plastic, an elastomer, a ceramic or metal. However, this device is about absorbing a liquid or semi-liquid from outside the applicator and applying the absorbed liquid to the skin. It is not designed for a liquid applicator in which liquid moves through the porous media and to an end for application to a porous surface.

U.S. Pat. No. 8,215,861 discloses a liquid flow through applicator with flocking on the applicator's surface, however, the applicator in this device does not contain a uniform porous media. This applicator uses a non-porous membrane with a few through orifices and flocking to deliver liquid through an inside reservoir to the outside of the flocking and onto the skin.

Liquid applicators and gel applicators should provide uniform liquid and gel delivery and a comfortable feeling when the applicators contact the skin. There is a need for improved liquid applicators and gel applicators over those disclosed in the prior art or in commercially available products.

### SUMMARY OF THE INVENTION

The present invention addresses this unmet need and provides a liquid applicator for applying a liquid or gel to a surface. The liquid applicator comprises a sintered porous elastomeric material body. Liquid moves from one end of the applicator through to another end of the liquid applicator by pushing the liquid through the applicator. In some embodiments, flocking is applied to an external end of the sintered porous elastomeric material body which contacts the surface for deposition of liquid.

In one embodiment, the sintered porous elastomeric material comprises a relatively rigid open end and a relatively flexible closed end. The flexible closed end is for surface contact and comprises a sintered porous elastomeric body with flocking on its external surface. The rigid open end is attached to the flexible end and also fits within an opening of a housing containing a fluid reservoir containing a liquid or gel. Upon application of pressure to the exterior walls of the reservoir, the fluid moves through the open end of the rigid component of the sintered porous elastomeric body and into the flexible closed end of the sintered porous elastomeric body. The fluid moves through the porous flexible closed end and is available for deposition onto a surface, such as skin.

In another embodiment, the sintered porous elastomeric material comprises a relatively rigid open end and a relatively flexible closed end. The flexible closed end is for surface contact and comprises a sintered porous elastomeric body without flocking on its external surface. The rigid open end is attached to the flexible end and also fits within an opening of a housing containing a fluid reservoir containing a liquid or gel. Upon application of pressure to the exterior walls of the reservoir, the fluid moves through the open end of the rigid component of the sintered porous elastomeric body and into the flexible closed end of the sintered porous elastomeric body. The fluid moves through the porous flexible closed end and is available for deposition onto a surface, such as skin.

Fluids which can be delivered to a surface include, without limitation, liquids, gels, emulsions and suspensions. These fluids may contain, without limitation, cosmetics and/or medicines.

### BRIEF DESCRIPTION OF THE FIGURES

FIG. 1. Cross-sectional drawing of a liquid applicator comprising a sintered elastomeric material body with flocking fibers on the end of the body.

FIG. 2. Cross-sectional drawing of a liquid applicator comprising a sintered elastomeric material body, wherein the sintered porous elastomeric body has two ends, one open end and one closed end with flocking fibers on the closed end.

FIG. 3. Cross-sectional drawing of a liquid applicator comprising a sintered elastomeric material body, wherein the sintered porous elastomeric body has two ends, one open end and one closed end. The open end is more rigid and has a smaller pore size than the closed end which is more flexible. Flocking fibers are on the closed end which is used to contact a surface for fluid delivery.

FIG. 4. Picture of a liquid application device comprising a liquid applicator with a porous sintered elastomeric body with flocking fibers on its exposed tip and a compressible tube.

FIG. 5. Flow rates (ml/min) of three different sintered porous liquid applicators with a similar shape as in FIG. 3.

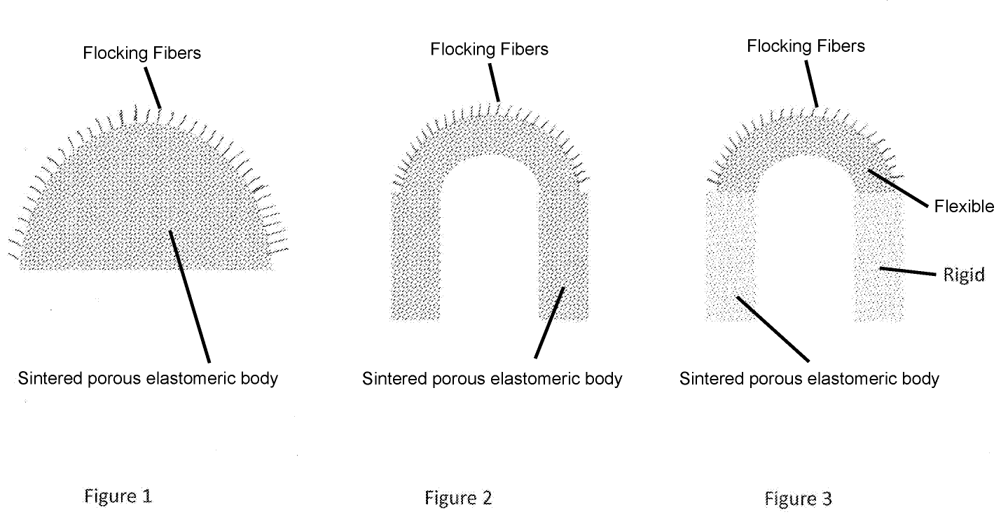

### DETAILED DESCRIPTION OF THE INVENTION

The present invention provides a liquid applicator for applying liquid to a surface and comprises a sintered porous elastomeric material body with or without flocking fibers on an external surface of the body.

In one embodiment the present invention provides a liquid applicator for applying liquid to a surface comprising a sintered porous elastomeric material body wherein the sintered porous elastomeric body has an average pore size greater than 20 microns, greater than 40 microns, greater than 60 microns, greater than 80 microns, greater than 100 microns, greater than 125 microns, greater than 150 microns, greater than 175 microns, greater than 200 microns, or greater than 250 microns. In some embodiments, the sintered porous elastomeric body has an average pore size less than about 300 microns.

In various embodiments, the elastomers used to make the sintered porous elastomeric material body can be selected from the group consisting of hydrogenated styrenic block copolymers, such as Septon® from Kuraray Co., Ltd. (Pasadena, Tex.); co-polyester based elastomers, such as Hytrel® from DuPont (Wilmington, Del.) and Arnitel from DSM (Troy, Mich.); styrene-butadiene-styrene block copolymers, such as Kraton® from Kraton Corporation (Houston, Tex.), Solprene from Dynasol (Houston, Tex.) and Dryflex® from Hexpol (Sandusky, Ohio); copolymer of ethylene-octene, such as Engage® from Dow Chemical (Midland Mich.); thermoplastic polyurethane such as Irogran®, Avalon®, Krystalgran®, and Irostic® from Huntsman (The Woodlands, Tex.), Desmopan®, Texin®, Desmoflex® and Desmovit® from Covestro (Pittsburgh, Pa.), Elastollan® from BASF (Florham Park, N.J.) and Estane®, Estloc™, and Pearthane™ from Lubrizol (Breckville, Ohio); silicone based elastomers, such as TPSiV® from Dow Corning (Midland, Mich.), ethylene-vinyl-acetate (EVA), such as Elevate® from Westlake Chemical (Houston, Tex.) and polypropylene based elastomer, such as Vistamaxx from ExxonMobile (Spring, Tex.). Other elastomeric materials known to one of ordinary skill in the art may be used.

In various embodiments, the plastic particles used to make parts of the sintered porous elastomeric material body can be selected from the group consisting of ethylene vinyl acetate (EVA), polypropylene (PP), polyethylene (PE) for example high density polyethylene (HDPE), low density polyethylene (LDPE) or ultrahigh molecular weight polyethylene (UHMWPE). Other plastics may be used as known to one of ordinary skill in the art.

In different embodiments, the flocking may be nylon fibers, polyethylene fibers, polypropylene fibers, cotton fibers, rayon fibers, polyester fibers or polyacrylic fibers. The fibers are attached to the sintered porous elastomeric body with an adhesive. The adhesives are commonly used in a flocking process, such vinyl, polyurethane, ethylene vinyl acetate (EVA) and epoxy based adhesives. The fibers have a length from about 0.1 mm to about 5 mm, from about 0.5 mm to about 4 mm or from about 1 mm to about 3 mm.

In one embodiment, the liquids to be applied with the applicators of the present invention are cosmetic products and have a viscosity from 50 cps to 5000 cps, from 100 cps to 4000 cps or from 500 to 2000 cps. A variety of agents may be applied with these applicators such as sunscreen, lotions, sunburn treatments, whitening agents, tanning agents, moisturizers, eye drops, antiperspirants, deodorants, cosmetics including but not limited to foundation, eyeliner, eye shadow, foundation, lip gloss and various liquid cosmetics. In other embodiments, medicine may be applied with these applicators. Such medicines include, but are not limited to, antibiotics, antibacterials, antiseptics, antihelminthics, antifungals, anesthetics, steroids such as glucocorticoids, anti-inflammatories, psoriasis medicines, surgical glue, fingernail and toenail treatments, skin cancer treatments, wart removal agents, isopropanol, and eczema treatments.

In one embodiment, the sintered porous elastomeric body is made from underwater pelletized elastomeric particles. These underwater pelletized elastomeric particles have an average particle size from about 0.25 mm to about 3 mm.

In another embodiment, the sintered porous elastomeric body is made from cryogenic ground elastomeric particles. These cryogenic ground elastomeric particles have an average particle size from about 100 microns to about 1000 microns.

The sintered porous elastomeric material body is molded. The liquid applicator is a molded single piece with a curved end for application to the surface of the skin.

The sintered porous elastomeric material body is made by sintering elastomeric particles in a mold. Elastomeric particles may be used to make the flexible end and/or the rigid end of the sintered porous elastomeric material body. Plastic particles may be used in the rigid and/or flexible end of the sintered porous elastomeric material body. The shape of the mold can be any desired shape allowing for the facile and single-step production of liquid applicators according to embodiments of the present invention.

Elastomer particles, in some embodiments, have average sizes ranging from about 10 μm to about 3 mm. In another embodiment, elastomer particles have average sizes ranging from about 20 μm to about 2 mm, from about 50 μm to about 1.5 mm, or from about 100 μm to about 1 mm.

Elastomer particles and plastic particles, in some embodiments, are sintered at a temperature ranging from about 93° C. to about 371° C. In some embodiments, plastic and elastomer particles are sintered at a temperature ranging from about 149° C. to about 260° C. The sintering temperature, according to embodiments of the present invention, is dependent upon and selected according to the identity of the plastic and elastomer particles.

Elastomer particles and plastic particles, in some embodiments, are sintered for a time period ranging from about 30 seconds to about 30 minutes. In other embodiments, plastic and elastomer particles are sintered for a time period ranging from about 1 minute to about 15 minutes or from about 5 minutes to about 10 minutes. In some embodiments, the sintering process comprises heating, soaking, and/or cooking cycles. Moreover, in some embodiments, sintering of plastic and elastomer particles is conducted under ambient pressure (1 atm). In other embodiments sintering of plastic and elastomer particles is conducted under pressures greater than ambient pressure.

In one embodiment, a liquid applicator for applying a liquid to a surface comprises a sintered porous elastomeric body, wherein the sintered porous elastomeric body comprises a relatively rigid end and a relatively flexible end. The flexible end is for surface contact and comprises a sintered porous elastomeric body and optionally has flocking on its external surface.

In another embodiment, a liquid applicator for applying a liquid to a surface comprises a sintered porous elastomeric body with two ends and a hollowed structure, wherein the sintered porous elastomeric body comprises a rigid open end and a flexible closed end. The flexible end is for surface contact and comprises a sintered porous elastomeric body and optionally has flocking on its external surface.

In another embodiment, a liquid applicator for applying a liquid to a surface comprises a sintered porous elastomeric body, wherein the sintered porous body comprises a relatively rigid end and a relatively flexible end. The relatively rigid end has a smaller average pore size than the pore size of the relatively flexible end. The relatively flexible end is for surface contact and comprises a sintered porous elastomeric body and optionally has flocking on its external surface. The relatively rigid end is for contact with a liquid container, such as a tube. Generally, the relatively flexible end has an average pore size greater than 20 microns, greater than 40 microns, greater than 60 microns, greater than 80 microns, greater than 100 microns, or greater than 150 microns. Generally, the relatively rigid end has an average pore size from about 20 microns to about 100 microns. The average pore size of the relatively rigid end is about 20 microns to about 100 microns smaller than the relatively flexible end. The hardness for the relatively flexible end ranges from about Shore OO 30 to about Shore A 80. The hardness for the relatively rigid end ranges from about Shore A 70 to about Shore D 50. The difference in hardness for a relatively flexible end and a relatively rigid end of a sintered porous elastomeric body are greater than 20 in the same Shore scale. For example, if the relatively flexible end had a hardness of Shore A 20, then the minimum hardness for the relatively rigid end will be at least Shore A 40.

Different combinations of elastomeric particles and/or plastic particles may be used to make the relatively rigid end and the relatively flexible end of the sintered porous elastomeric body. Elastomers used to make the sintered porous elastomeric material body can be selected from the group consisting of hydrogenated styrenic block copolymers, such as Septon® from Kuraray Co., Ltd. (Pasadena, Tex.); co-polyester based elastomers, such as Hytrel® from DuPont (Wilmington, Del.) and Arnitel from DSM (Troy, Mich.); styrene-butadiene-styrene block copolymers, such as Kraton® from Kraton Corporation (Houston, Tex.), Solprene from Dynasol (Houston, Tex.) and Dryflex® from Hexpol (Sandusky, Ohio); copolymer of ethylene-octene, such as Engage® from Dow Chemical (Midland Mich.); thermoplastic polyurethane such as Irogran®, Avalon®, Krystalgran®, and Irostic® from Huntsman (The Woodlands, Tex.), Desmopan®, Texin®, Desmoflex® and Desmovit® from Covestro (Pittsburgh, Pa.), Elastollan® from BASF (Florham Park, N.J.) and Estane®, Estloc™, and Pearthane™ from Lubrizol (Breckville, Ohio); silicone based elastomers, such as TPSiV® from Dow Corning (Midland, Mich.), ethylene-vinyl-acetate (EVA), such as Elevate® from Westlake Chemical (Houston, Tex.) and polypropylene based elastomer, such as Vistamaxx from ExxonMobile (Spring, Tex.). Other elastomeric materials known to one of ordinary skill in the art may be used.

Plastic particles can be selected from the group consisting of ethylene vinyl acetate (EVA), polypropylene (PP), polyethylene (PE) for example high density polyethylene (HDPE), low density polyethylene (LDPE) or ultrahigh molecular weight polyethylene (UHMWPE).

In some embodiments, the following non-limiting combinations of elastomeric particles and plastic particles may be employed to make the sintered porous elastomeric body comprising a relatively flexible end and a relatively rigid end: SBC and UHMWPE; SBC and HDPE; SBC and LDPE. SBC and PP; SBC and EVA; TPU and UHMWPE; TPU and HDPE; TPU and LDPE; TPU and PP; TPU and EVA. In one embodiment, the relatively flexible end and the relatively rigid end are made from elastomeric particles and the elastomeric particles in the relatively flexible end are softer than the elastomeric particles in the relatively rigid end.

In another embodiment, the relatively flexible end is made from elastomeric particles and the relatively rigid end is made from elastomeric particles and plastic particles.

In yet another embodiment, the relatively flexible end is made from elastomeric particles and the relatively rigid end is made from plastic particles.

In still another embodiment, both the relatively flexible end and the relatively rigid end are made from elastomeric particles and plastic particles, wherein the relatively rigid end's elastomeric particle to plastic particle weight ratio is lower than that of the relatively flexible end.

The sintered liquid applicator with a relatively rigid end and a relatively flexible end are made by one step sintering process. The typical sintering processes were described in U.S. Pat. No. 8,141,717.

The sintered liquid applicator with a relatively flexible end and a relatively rigid end is made by sintering particles or a mixture of particles in a mold. The shape of the mold can be any desired shape allowing for the facile and single-step production of liquid applicators according to embodiments of the present invention.

In one embodiment, a method for producing a liquid applicator with a relatively flexible end and a relatively rigid end comprises disposing a first set of elastomeric particles in a first part of a mold cavity, disposing a second set of elastomeric particles in a second part of the mold cavity adjacent to the first part of the mold cavity, and sintering the particles into a sintered porous product.

In another embodiment, a method for producing a liquid applicator with a relatively flexible end and a relatively rigid end comprises disposing elastomeric particles in a first part of a mold cavity, disposing plastic particles in a second part of the mold cavity adjacent to the first part of the mold cavity, and sintering the particles into a sintered porous product.

In yet another embodiment, a method for producing a liquid applicator with a relatively flexible end and a relatively rigid end comprises disposing a first mixture of elastomeric particles and plastic particles in a first part of a mold cavity, disposing a second mixture of elastomeric particles and plastic particles in a second part of the mold cavity adjacent to the first part of the mold cavity, and sintering the particles into a sintered porous product.

In another embodiment, a method for producing a liquid applicator with a relatively flexible end and a relatively rigid end comprises disposing a first mixture of elastomeric particles and plastic particles in a first part of a mold cavity, plastic particles in a second part of the mold cavity adjacent to the first part of the mold cavity, and sintering the particles into a sintered porous product.

Elastomer and plastic particles for the relatively flexible end, in some embodiments, have average sizes ranging from about 10 μm to about 3 mm. In other embodiments, elastomeric particles and plastic particles have average sizes ranging from about 20 μm to about 2 mm, from about 50 μm to about 1.5 mm, or from about 100 μm to about 1 mm.

Elastomeric and plastic particles for the relatively rigid end, in some embodiments, have average sizes ranging from about 10 μm to about 2 mm. In other embodiments, elastomeric particles and plastic particles have average sizes ranging from about 20 μm to about 1.5 mm, from about 50 μm to about 1 mm, or from about 100 μm to about 800 μm.

The average particle size in the relatively flexible end is larger than the average particle size in relatively rigid end. The average particle size in the relatively flexible end is from about 20 microns to 200 microns larger than the average particle size in the relatively rigid end.

Elastomeric and plastic particles, in some embodiments, are sintered at a temperature ranging from about 93° C. to about 371° C. In some embodiments, plastic and elastomeric particles are sintered at a temperature ranging from about 149° C. to about 260° C. The sintering temperature, according to embodiments of the present invention, is dependent upon and selected according to the identity of the plastic and elastomeric particles.

Elastomeric and plastic particles, in some embodiments, are sintered for a time period ranging from about 30 seconds to about 30 minutes. In other embodiments, plastic and elastomeric particles are sintered for a time period ranging from about 1 minute to about 15 minutes or from about 5 minutes to about 10 minutes. In some embodiments, the sintering process comprises heating, soaking, and/or cooking cycles. Moreover, in some embodiments, sintering of plastic and elastomeric particles is conducted under ambient pressure (1 atm). In other embodiments sintering of plastic and elastomeric particles is conducted under pressures greater than ambient pressure.

In yet another embodiment, a liquid applicator for applying a liquid to a surface comprises a sintered porous elastomeric body, wherein the sintered porous elastomeric body comprises a relatively rigid end and a relatively flexible end. The relatively rigid end has a smaller average pore size than the pore size of the relatively flexible end. The relatively flexible end is for surface contact and comprises a sintered porous elastomeric body and optionally has flocking on its external surface. The relatively flexible end has an average pore size greater than 20 microns, greater than 40 microns, greater than 60 microns, greater than 80 microns, greater than 100 microns, or greater than 150 microns. The average pore size of the relatively rigid end is about 20 microns to about 100 microns smaller than the relatively flexible end.

In another embodiment, the liquid applicator for applying a liquid to a surface comprises a sintered porous elastomeric body with two ends and a hollowed structure, wherein the sintered porous body comprises a relatively rigid open end and a relatively flexible closed end. The relatively rigid end has a smaller average pore size than the pore size of the relatively flexible end. The relatively flexible end is for surface contact and comprises a sintered porous elastomeric body and optionally has flocking on its external surface. The relatively flexible end has an average pore size greater than 20 microns, greater than 40 microns, greater than 60 microns, greater than 80 microns, greater than 100 microns, or greater than 150 microns. The average pore size of the relatively rigid end is about 20 microns to about 100 microns smaller than the relatively flexible end.

In another embodiment, a liquid applicator for applying a liquid to a surface comprises a sintered porous elastomeric body with two ends and a hollowed structure, wherein the sintered porous body comprises a relatively rigid open end and a relatively flexible closed end. The relatively rigid end has a smaller average pore size than the pore size of the relatively flexible end. The relatively flexible end is for surface contact and comprises a sintered porous elastomeric body and optionally has flocking on its external surface. The flexible end has an average pore size greater than 20 microns, greater than 40 microns, greater than 60 microns, greater than 80 microns, greater than 100 microns or greater than 150 microns. The average pore size of the relatively rigid end is about 20 microns to about 100 microns smaller than the relatively flexible end.

In one embodiment, a liquid application device assembly comprises a housing with an open and a closed end, the housing enclosing a liquid containing compartment, and a liquid applicator, wherein a first end of the liquid applicator is at the open end of the housing, and a second end of the liquid applicator is located inside the liquid compartment within the opening of the housing. In one embodiment, the second end of the liquid applicator can fit within the opening of the fluid reservoir through a frictional fit. In another embodiment, the second end of the liquid applicator is threaded on its external surface and can fit within the opening of the fluid reservoir by screwing the second end into a threaded inner wall of the opening. In yet another embodiment, the second end of the liquid applicator can be glued within the opening of the fluid reservoir on its inner wall. In another embodiment, the second end of the liquid applicator contains a circumferential ridge on its outer surface and can be snapped into a slot in the inner wall of the opening of the fluid reservoir.

Liquid inside the liquid compartment moves through the liquid applicator and to the first end of the liquid applicator. The first end of the liquid applicator is placed in contact with a surface, such as skin, for application of the liquid to the surface.

In another embodiment, a liquid application device assembly comprises a housing with an open and a closed end, a liquid containing compartment, and a liquid applicator, wherein a first end of the liquid applicator is at the open end of the housing, and a second end of the liquid applicator is located inside the liquid compartment. Liquid inside the liquid compartment moves through the liquid applicator and to the first end of the liquid applicator which optionally has flocking on the external surface of the first end. The first end of the liquid applicator is placed in contact with the skin for application of the liquid. Most of the fluid moves through the open end of the liquid applicator although some fluid may move through the porous relatively rigid end into the relatively flexible end.

In yet another embodiment, a liquid application device assembly comprises a housing with an open and a closed end, a liquid containing compartment, and a liquid applicator, wherein a first end of the liquid applicator is at the open end of the housing, and a second end of the liquid applicator is located inside the liquid compartment. Liquid inside the liquid compartment moves through the liquid applicator and to the first end of the liquid applicator which optionally has flocking on the external surface of the first end. The first end of the liquid applicator is placed in contact with the skin for application of the liquid. The sintered porous elastomeric material has an average pore size greater than 20 microns, greater than 40 microns, greater than 60 microns, greater than 80 microns, greater than 100 microns or greater than 150 microns. These pore sizes can be for both the relatively flexible and for the relatively rigid end of the liquid applicator although the relatively rigid end has smaller pore sizes than the relatively flexible end by at least 20 microns.

Sintered Porous Elastomeric Material

The sintered porous elastomeric material has an average pore size from about 20 microns to about 300 microns. The sintered porous elastomeric material has an average porosity of at least 15%. The elastomeric particles that form the sintered porous elastomeric applicator are made by underwater pelletizing and with an average particle size from about 0.25 mm to about 2.5 mm. In another embodiment, the elastomeric particles that form the sintered porous elastomeric applicator are made from cryogenic ground elastomeric particles. These cryogenic ground elastomeric particles have an average particle size from about 100 microns to about 1000 microns.

The sintered porous elastomeric material has an average hardness between Shore OO 30 to Shore A 80. The sintered porous elastomeric material made from ground particles has an average hardness between Shore OO 30 to Shore A 50. The sintered porous elastomeric material made from underwater pelletized particles has an average hardness between Shore A 10 to about Shore A 80.

The hardness for the relatively flexible end ranges from about Shore OO 30 to about Shore A 80. The hardness for the relatively rigid end range from about Shore A 70 to about Shore D 50. The difference in hardness for a sintered porous elastomeric body with a relatively flexible end and a relatively rigid end are greater than 20 in the same Shore scale. For example, if the relatively flexible end had a hardness of Shore A 20, then the minimum hardness for the relatively rigid end will be at least Shore A 40.

In various embodiments, the elastomers used to make the sintered porous elastomeric material body can be selected from the group consisting of hydrogenated styrenic block copolymers, such as Septon® from Kuraray Co., Ltd. (Pasadena, Tex.); co-polyester based elastomers, such as Hytrel® from DuPont (Wilmington, Del.) and Arnitel from DSM (Troy, Mich.); styrene-butadiene-styrene block copolymers, such as Kraton® from Kraton Corporation (Houston, Tex.), Solprene from Dynasol (Houston, Tex.) and Dryflex® from Hexpol (Sandusky, Ohio); copolymer of ethylene-octene, such as Engage® from Dow Chemical (Midland Mich.); thermoplastic polyurethane such as Irogran®, Avalon®, Krystalgran®, and Irostic® from Huntsman (The Woodlands, Tex.), Desmopan®, Texin®, Desmoflex® and Desmovit® from Covestro (Pittsburgh, Pa.), Elastollan® from BASF (Florham Park, N.J.) and Estane®, Estloc™, and Pearthane™ from Lubrizol (Breckville, Ohio); silicone based elastomers, such as TPSiV® from Dow Corning (Midland, Mich.), ethylene-vinyl-acetate (EVA), such as Elevate® from Westlake Chemical (Houston, Tex.) and polypropylene based elastomer, such as Vistamaxx from ExxonMobile (Spring, Tex.). Other elastomeric materials known to one of ordinary skill in the art may be used.

In another embodiment, the elastomers used to make the sintered porous elastomeric material body are thermoplastic urethane (TPU). TPUs includes aromatic polyester based TPU, aromatic polyether based TPU and aliphatic TPU.

In some embodiments, TPUs used to make the sintered porous elastomeric material body are aromatic polyether based TPUs. Aromatic TPUs include toluene diisocyanate (MI) and methylenediphenyl diisocyanate (MDI) based TPU.

Aliphatic TPUs include hexamethylene diisocyanate (HDI), methylene dicyclohexyl diisocyanate or hydrogenated. MDI (HMDI) and isophorone diisocyanate based TPU.

Polyester based TPUs include TPUs containing polyol made from diacids and glycols.

Polyether based TPUs include TPUs containing polyether made from ethylene oxide, propylene, oxide or tetrahydrofuran.

In various embodiments, the plastic particles used to make parts of the sintered porous elastomeric material body can be selected from the group consisting of ethylene vinyl acetate (EVA), polypropylene (PP), polyethylene (PE) for example high density polyethylene (HDPE), low density polyethylene (LDPE) or ultrahigh molecular weight polyethylene (UHMWPE). Other plastics may be used as known to one of ordinary skill in the art.

In one embodiment sintered porous elastomeric material comprises antimicrobial agents.

In another embodiment, at least part of elastomeric particles in the sintered porous elastomeric material comprise antimicrobial agents.

The optional flocking fibers are attached to the sintered porous elastomeric material at an angle of about 90 degrees.

In one embodiment the housing is a flexible housing and can be compressed by hand.

In another embodiment the housing is rigid and has a mechanical pushing mechanism, such as a screw or a spring.

The liquid applicator of the present invention could be used in the applicator devices described in the following patents; U.S. Pat. Nos. 8,215,861, 8,141,717, 8,168,262, 8,114,027, 7,955,018, 7,874,300, 7,722,276, 7,957,459, 7,040,827, 6,840,694, 6,773,187, 6,715,951, 6,638,067, 6,634,821, 6,283,664, 6,096,382 or U.S. Pat. No. 5,567,073.

The following examples will serve to further illustrate the present invention without, at the same time, however, constituting any limitation thereof. On the contrary, it is to be clearly understood that resort may be had to various embodiments, modifications and equivalents thereof which, after reading the description herein, may suggest themselves to those skilled in the art without departing from the spirit of the invention.

### Example 1

Liquid Applicator with Flocking and a Sintered Porous Elastomer of Styrenic Block Co-Polymer

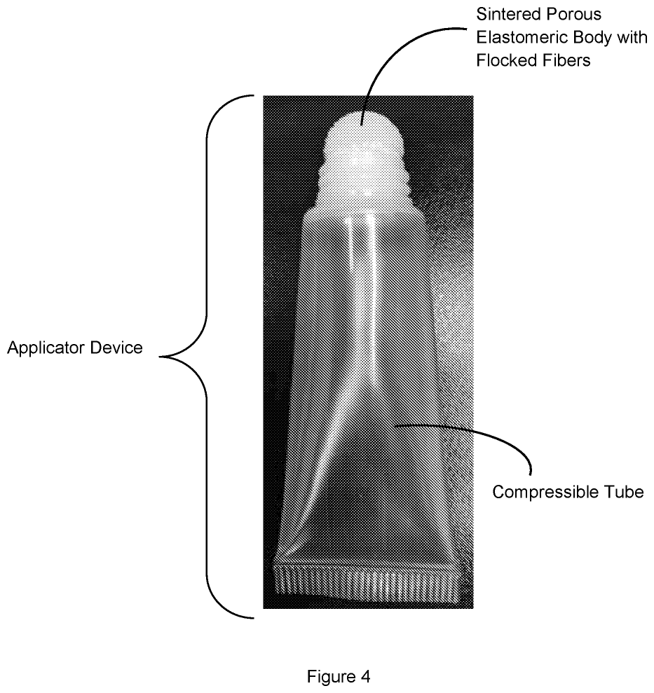

A 3-dimensional applicator device having two components is exemplified in FIG. 4. The applicator has a top sintered porous elastomeric component and a bottom component which is a compressible tube with a fluid reservoir inside.

Top Sintered Porous Elastomeric Component

The sintered porous elastomeric component had a shape as shown in FIG. 3. The relatively flexible dome shape was made from porous plastic hydrogenated styrenic block co-polymer (SBC). This component had a 170 micron pore size and 33% pore volume. The outer surface of the relatively flexible dome shaped part was then flocked with 1.0 mm 1.7 decitex (dtex—mass in grams per 10,000 meters) PA6.6 nylon fibers using a polyurethane adhesive. The relatively rigid part which fit into the opening of the tube was made from ethylene vinyl acetate (EVA). The EVA component had an average pore size of about 80 microns and 20% pore volume. The EVA particles and the SBC particles were placed in different regions of a mold and were sintered. The hardness of the relatively flexible end was about Shore A 10 and the hardness of the relatively rigid end was about Shore A 80.

Bottom Component

The bottom component was a compressible tube made from polypropylene with a fluid reservoir containing silicon oil (1 Pa·s (pascal second) viscosity which equals 1000 cP (centipoise)).

Upon application of pressure to the compressible tube containing silicone oil, silicone oil flowed from the liquid reservoir and into and through the sintered porous elastomeric component for release from the flexible dome shaped part with flocking fibers onto a surface, such as skin.

### Example 2

Liquid Applicator with Sintered Porous Thermoplastic Polyurethane Elastomer

A 3-dimensional applicator device having two components is exemplified in FIG. 4. The applicator has a top sintered porous component and a bottom component which is a compressible tube with a liquid reservoir inside.

Top Sintered Porous Elastomeric Component

The sintered porous component had a shape as shown in FIG. 3 but without flocking fibers. The relatively flexible dome shape was made from ground thermoplastic polyurethane (TPU). This component had a 140 micron pore size and 52% pore volume. The relatively rigid part which fit in the opening of the tube was made from sintered porous ultrahigh molecular weight polyethylene (UHMWPE) with an average pore size of 30 microns and pore volume of about 40%. The UHMWPE particles and TPU particles were placed in different regions of a mold and were sintered. The hardness of the relatively flexible end was about Shore A 10 and the hardness of the relatively rigid end was about Shore A 90.

Bottom Component

The bottom component was a compressible tube containing a fluid reservoir with silicon oil (1 Pa·s viscosity). Upon application of pressure to the compressible tube, silicone oil flowed from the liquid reservoir, into and through the sintered porous elastomeric component for release from the flexible dome shaped part onto a surface, such as skin.

### Example 3

Liquid Applicator with Sintered Porous Thermoplastic Polyurethane Elastomer

A 3-dimensional applicator device having two components is exemplified in FIG. 4. The applicator has a top sintered porous component and a bottom component which is a compressible tube with a liquid reservoir inside.

Top Sintered Porous Component

The sintered porous component had a shape as shown in FIG. 3, but without flocking fibers. The relatively flexible dome shaped part was made from underwater pelletized thermoplastic polyurethane (TPU). This part had a 190 micron pore size and 20% pore volume. The relatively rigid part which fit in the opening of the tube was made from sintered porous UHMWPE with average pore size of 30 microns and pore volume of about 40%. The UHMWPE particles and TPU particles were placed in different regions of a mold and were sintered. The hardness of the relatively flexible end was about Shore A 30 and the hardness of the relatively rigid end was about Shore A 90.

Bottom Component

The bottom component was a compressible tube containing silicon oil. Upon application of pressure to the compressible tube (1 Pa·s viscosity), silicone oil flowed from the liquid reservoir, into and through the sintered porous elastomeric component for release from the flexible dome shaped part onto a surface, such as skin.

### Example 4

Liquid Applicator with Sintered Porous Thermoplastic Polyurethane Elastomer and Nylon Flocking

A 3-dimensional applicator device having two components is exemplified in FIG. 4. The applicator has a top sintered porous thermoplastic elastomeric component and a bottom component which is a compressible tube with a liquid reservoir inside.

Top Sintered Porous Elastomeric Component

The sintered porous elastomeric component has a shape as shown in FIG. 3. The relatively flexible dome shaped part is made from ground aromatic polyether based thermoplastic urethane (TPU). This part has a 140 micron pore size and 52% pore volume. This dome shaped part is then flocked on its outer surface with 1.0 mm 1.7 decitex (dtex—mass in grams per 10,000 meters) PA6.6 nylon fibers using a polyurethane adhesive. The relatively rigid part which fits in the opening of the tube is made from ethylene vinyl acetate (EVA). The EVA component has an average pore size of about 80 microns and 20% pore volume. The TPU particles and the EVA particles are placed in different regions of a mold and are sintered.

Bottom Component

The bottom component is a compressible tube containing silicon oil. Upon application of pressure to the compressible tube (1 Pa·s viscosity), silicone oil flows from the liquid reservoir and into and through the sintered porous elastomeric component for release from the flexible dome shaped part with flocking fibers onto a surface, such as skin.

### Example 5

Liquid Applicator with Sintered Porous Thermoplastic Polyurethane Elastomer

A 3-dimensional applicator device having two components is exemplified in FIG. 4. The applicator has a top sintered porous elastomeric component and a bottom component which is a compressible tube with a liquid reservoir inside.

Top Sintered Porous Elastomeric Component

The sintered porous elastomeric component has a shape as shown in FIG. 3. The relative flexible dome shaped part is made from underwater pelletized aromatic polyether based thermoplastic urethane (TPU). This component has an average pore size of 190 microns and 20% pore volume. The relatively rigid part which fits in the opening of the tube is made from sintered ethylene vinyl acetate (EVA). The EVA component has an average pore size of about 80 microns and 20% pore volume. The TPU particles and the EVA particles are placed in different regions of a mold and are sintered.

Bottom Component

The bottom component was a compressible tube containing silicon oil (1 Pa·s viscosity). Upon application of pressure to the compressible tube, silicone oil flows from the liquid reservoir, and into and through the sintered porous elastomeric component for release from the flexible dome shaped part onto a surface, such as skin.

### Example 6

Solvent Stability of Sintered Porous Polyurethane

Sintered porous thermoplastic polyurethane used in embodiments described herein are stable in solvents used in the cosmetic industry. Table 1 lists properties of sintered porous thermoplastic urethane before and after immersion in different solvent for 24 hours. The parts were tested in dry conditions. The parts were made from two types of TPU particles, ground particles and underwater pelletized particles. The sintered TPU (both ground particles and underwater pelletized particles) showed outstanding stability in deionized water, isopropanol (IPA) and n-decane.

TABLE 1
Sintered TPU solvent stability

| | | | | | | Ariflow | Airflow | |
|---|---|---|---|---|---|---|---|---|
| | | | | | | Before | After | Tear |
| | | OD | OD | Thickness | Thickness | ml/min | ml/min | resistance |
| | Parameter | before | after | Before | After | @ 1.2″ | @ 1.2″ | before |
| Solvent | Unit | inch | inch | inch | inch | H2O | H2O | psi |
| IPA | Ground | 1.0545 | 1.048 | 0.1215 | 0.1195 | 5743 | 5378 | 3.264 |
| | Micro-pellet | 1.052 | 1.0545 | 0.122 | 0.1195 | 5597 | 5305 | 31.28 |
| DI water | Ground | 1.048 | 1.0495 | 0.1225 | 0.124 | 5597 | 5524 | 3.264 |
| | Micro-pellet | 1.062 | 1.0585 | 0.123 | 0.122 | 5597 | 5743 | 31.28 |
| N-Decane | Ground | 1.0555 | 1.0555 | 0.1195 | 0.121 | 5670 | 5670 | 3.264 |
| | Micro-pellet | 1.057 | 1.0655 | 0.1215 | 0.1255 | 5670 | 5524 | 31.28 |

| | | Tear | | | | | pore | pore |
|---|---|---|---|---|---|---|---|---|
| | | resistance | tensile | tensile | pore size | pore size | volume | volume |
| | Parameter | after | before | after | before | after | before | after |
| Solvent | Unit | psi | psi | psi | um | um | % | % |
| IPA | Ground | 2.944 | 12.48 | 11.52 | 140.23 | 125.3 | 50.44 | 48.41 |
| | Micro-pellet | 32.08 | 22.64 | 25.12 | 195.46 | 189.78 | 19.79 | 18.52 |
| DI water | Ground | 3 | 12.48 | 15.52 | 140.23 | 129.27 | 50.44 | 52.21 |
| | Micro-pellet | 35.12 | 22.64 | 24.48 | 195.46 | 191.72 | 19.79 | 18.03 |
| N-Decane | Ground | 2.864 | 12.48 | 14.88 | 140.23 | 137.49 | 50.44 | 48.3 |
| | Micro-pellet | 30 | 22.64 | 23.52 | 195.49 | 194.46 | 19.79 | 20.92 |

### Example 7

Flow Properties of Sintered Porous Hydrogenated Styrenic Block Co-Polymer (SBC) Material for Silicon Oil at Different Viscosities.

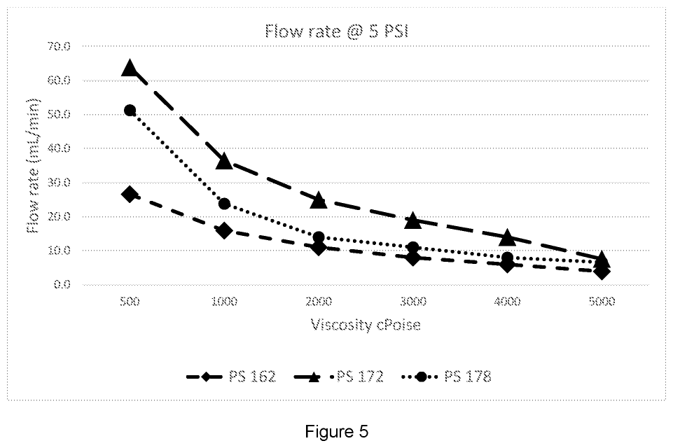

Sintered porous liquid applicators with the shape of FIG. 3, without flocking fibers, were tested at 5 psi pressure for silicone oil with different viscosities. The relatively flexible part was made from hydrogenated styrenic block co-polymer (SBC) particles. The relatively rigid part which fit into the opening of the tube was made from ethylene vinyl acetate (EVA) particles. The EVA component had an average pore size of about 80 microns and 20% pore volume. The SBC particles and the EVA particles were placed in different regions of a mold and were sintered. The liquid applicators had a dome diameter of about 12 mm and a wall thickness of 3 mm. PS 162 had an average pore size of 162 microns and a pore volume of about 49% and was made from ground SBC particles and EVA particles. PS 172 had an average pore size of 172 microns and pore volume of about 19% and was made from underwater pelletized SBC particles and EVA particles. PS 178 had an average pore size of 178 microns and pore volume of about 33% and was made from underwater pelletized SBC particles and EVA particles. FIG. 5 shows that sintered SBC-based porous elastomer delivers good liquid flow from low viscosity to high viscosity.

All patents, publications and abstracts cited above are incorporated herein by reference in their entirety. It should be understood that the foregoing relates only to preferred embodiments of the present invention and that numerous modifications or alterations may be made therein without departing from the spirit and the scope of the present invention as defined in the following claims.

1. A liquid applicator comprising a sintered porous elastomeric material body comprising a first end and a second end, wherein the first end comprises a relatively flexible region and the second end comprises a relatively rigid region.

2. The liquid applicator of claim 1, wherein the sintered porous elastomeric material body has flocking fibers on the first end.

3. The liquid applicator of claim 1, wherein the relatively rigid end of the sintered porous elastomeric material body is hollow.

4. The liquid applicator of claim 1, wherein the relatively rigid end of the sintered porous elastomeric material body is for coupling to a housing.

5. The liquid applicator of claim 1, wherein the relatively flexible end of the sintered porous elastomeric material body is for contacting a surface.

6. The liquid applicator of claim 1, wherein the sintered porous elastomeric material body comprises an elastomer selected from the group consisting of hydrogenated styrenic block copolymers, co-polyester based elastomers, styrene-butadiene-styrene block copolymers, copolymer of ethylene-octene, thermoplastic polyurethane, silicone based elastomers, ethylene vinyl acetate based elastomers and polypropylene based elastomers.

7. The liquid applicator of claim 1, wherein the sintered porous elastomeric material body comprises an plastic selected from the group consisting of ethylene vinyl acetate (EVA), polypropylene (PP), and polyethylene (PE) for example high density polyethylene (HDPE), low density polyethylene (LDPE) or ultrahigh molecular weight polyethylene (UHMWPE).

8. The liquid applicator of claim 1, wherein the sintered porous elastomeric material body comprises a sintered porous thermoplastic polyurethane elastomeric material.

9. The liquid applicator of claim 8, wherein the thermoplastic polyurethane elastomeric material is an aromatic, polyether-based thermoplastic polyurethane.

10. The liquid applicator of claim 2, wherein the flocking fibers are selected from the group consisting of nylon fibers, polyethylene fibers, polypropylene fibers, cotton fibers, rayon fibers, polyester fibers and polyacrylic fibers.

11. The liquid applicator of claim 1, wherein the relatively flexible end of the sintered porous elastomeric material body is made from one or more elastomers.

12. A device for applying a liquid or a gel to a surface comprising:
a housing with a closed end and an open end;
a fluid reservoir in the housing; and,
the liquid applicator of any of the preceding claims, wherein the second end is located in the fluid reservoir and the first end is located at or near the open end of the housing.

13. A method of applying a liquid or a gel to a surface comprising:
providing the device of claim 12;
applying the second end of the liquid applicator to the surface;
compressing the housing; and,
applying the liquid or the gel from the first end of the liquid applicator to the surface.

14. The method of claim 13, wherein the surface is skin.

15. The liquid or the gel of claim 13, wherein liquid or the gel is a cosmetic or a medicine.
